(19) United States
(12) Patent Application Publication
(10) Pub. No.: US 2013/0302267 A1
(43) Pub. Date: Nov. 14, 2013

(54) HAIR TREATMENT COMPRISING SILICONE GRAFTED STARCH

(71) Applicant: THE PROCTER & GAMBLE COMPANY, Cincinnati, OH (US)

(72) Inventors: Marjorie Mossman Peffly, Cincinnati, OH (US); Kendrick Jon Hughes, Sycamore Township, OH (US); David Frederick Swaile, Cincinnati, OH (US)

(73) Assignee: The Procter & Gamble Company, Cincinnati, OH (US)

(21) Appl. No.: 13/891,473

(22) Filed: May 10, 2013

Related U.S. Application Data
(60) Provisional application No. 61/645,744, filed on May 11, 2012.

(51) Int. Cl. A61K 8/91; A61Q 5/06; A61Q 5/12; A61Q 5/00

(52) U.S. Cl. CPC A61K 8/91; A61Q 5/00; A61Q 5/06; A61Q 5/12
USPC 424/70.121

(57) ABSTRACT

Hair treatment compositions that can increase the appearance of thicker and fuller hair, by increasing hair volume and root lift. More specifically, it relates to a hair treatment or styling composition including a silicone grafted starch and a film forming polymer. Such compositions can be applied to any areas where a thicker and fuller hair appearance is desired, such as the hair and/or scalp.

### FIELD OF THE INVENTION

The present invention relates to a hair treatment or styling composition and methods of making and using the same. More specifically, it relates to a hair treatment or styling composition including a silicone grafted starch and a film forming polymer.

### BACKGROUND OF THE INVENTION

Historically it has been difficult to provide a leave on hair treatment and/or styling composition which delivers both hair volume and root lift without resulting in negative hair feel tradeoffs, such as dry hair, stickiness or lack of conditioning. In leave-on compositions a number of attempts have been made to address aspects of volume or hair feel with limited success. For creation of hair volume for example, the addition of solid particles to hair care compositions to separate hair fibers, including smooth round particles polyethylene with irregular shaped silica particles to help minimize the hair feel tradeoff, however a negative hair feel is associated with these compositions. To further improve hair feel, a separate silicone can be added to volumizing compositions. However, the addition of silicones can result in a tradeoff in the root lift of volume benefit of the composition and may further result in a greasy feeling to the hair. Additionally, when a film forming polymer is used, the presence of silicone may affect adhesion of the film forming polymers to the hair. Depending on the solubility/compatibility of the silicone, the separately added silicone can also plasticize the film forming polymer which can reduce the film integrity under high humidity conditions, which can reduce style hold.

It has been found that providing a silicone graft to the dry particles (solid) results in a better hair feel without trading off volume. The volume and hair feel benefits are further improved in combination with a film forming polymer, resulting in a hair volumizing composition that has good adhesion to the hair and good humidity resistance.

In particular, the combination of silicone grafted starch, such as tapioca, plus a film forming polymer results in desirable root lift/volume and good hair feel combinations. Additionally, this combination minimizes visible residue that may occur with other particle types. A good combination of benefits is especially realized in using silicone grafted tapioca starch when it is used at a level from about 0.1% to about 5% in combination with a film forming polymer used at levels of from about 0.1% to about 5%.

### SUMMARY OF THE INVENTION

A hair treatment composition comprising from about 0.01% to about 5% by weight of the composition of a silicone grafted tapioca starch, from about 0.01 to about 5% by weight of the composition of a film forming polymer, and a carrier.

### BRIEF DESCRIPTION OF THE DRAWINGS

While the specification concludes with the claims particularly pointing out and distinctly claiming the invention, it is believed that the present invention will be better understood from the following description taken in conjunction with the accompanying drawings in which:

FIG. 1 is a front view of the switch cart showing the ARC points;

FIG. 2 is a front view of the switch cart showing the ARC points;

FIG. 3 is a front view of the switch cart showing the ARC points;

FIG. 4 graph showing data points for compositions.

### DETAILED DESCRIPTION OF THE INVENTION

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M·Wt.” as used herein refers to the weight average molecular weight unless otherwise stated. “QS” means sufficient quantity for 100%.

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights percents (wt %) as they pertain to listed ingredients are based on the active level and do not include carriers or by-products that may be included in commercially available materials, unless otherwise specified.

Herein, “comprising” means that other steps and other ingredients which do not affect the end result can be added. This term encompasses the terms “consisting of” and “consisting essentially of”. The compositions, methods, uses, kits, and processes of the present invention can comprise, consist of, and consist essentially of the elements and limitations of the invention described herein, as well as any of the additional or optional ingredients, components, steps, or limitations described herein.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, and/or alcohol derivatives of a given compound.

“Polymer,” as used herein, means a chemical formed from the polymerisation of two or more monomers, which may be the same or different. The term “polymer” as used herein shall include all materials made by the polymerisation of monomers as well as natural polymers. Polymers made from only one type of monomer are called homopolymers. A polymer comprises at least two monomers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

The term “charge density” as used herein, means the ratio of the number of positive charges on a monomeric unit of which a polymer is comprised to the M·Wt. of said monomeric unit. The charge density multiplied by the polymer M·Wt. determines the number of positively charged sites on a given polymer chain. For cationic guars, charge density is measured using standard elemental analysis of percentage nitrogen known to one skilled in the art. This value of percentage nitrogen, corrected for total protein analysis, can then be used to calculate the number or equivalence of positive charges per gram of polymer. For the cationic copolymers, the charge density is a function of the monomers used in the synthesis. Standard NMR techniques know to one skilled in the art would be used to confirm that ratio of cationic and non-ionic monomers in the polymer. This would then be used to calculate the number or equivalence of positive charges per gram of polymer. Once these values are know, the charge density is reported in milliequivalence (meq) per gram of cationic polymer.

The term “hair treatment composition” includes leave-on product compositions comprising a silicone grafted starch polymer and a film forming polymer. Film forming polymers could include viscosity-modifying substances, hair conditioning substances such as silicones or proteins, or hair-setting polymers. The hair treatment composition may further comprise at least one other non-polymeric active ingredients or additives chosen from scalp care substances, hair care substances, photoprotective substances, oils, waxes, preservatives, pigments, soluble dyes, particulate substances, and surfactants in a suitable cosmetic base. The hair-treatment composition can be in the form of a gel, a viscous lotion, a spray gel, an O/W emulsion, a W/O emulsion, a microemulsion, a hydroalcoholic composition, a hair spray, a foamable composition in combination with a device for foaming.

The features of the composition according to the first aspect, as well as the other aspects and other relevant components, are described in detail hereinafter. All components of the composition described herein should be physically and chemically compatible with the essential components described herein, and should not otherwise unduly impair composition stability, aesthetics or performance.

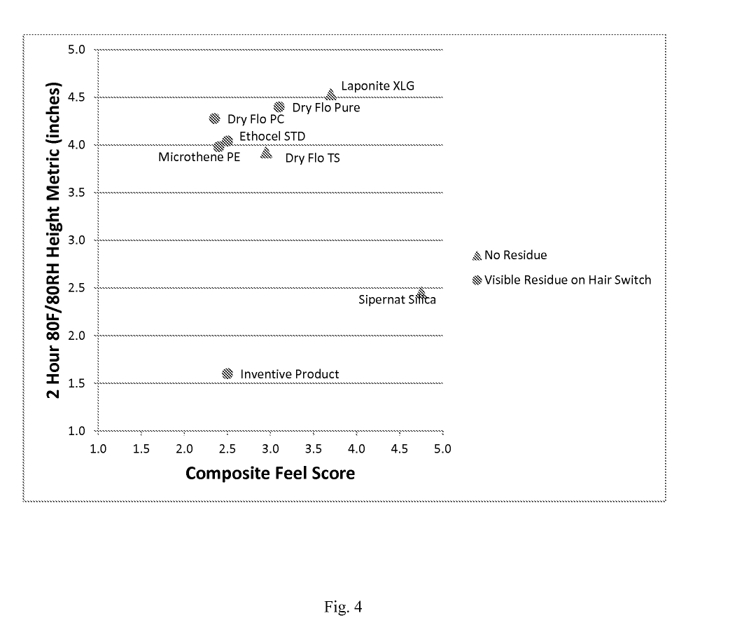

In accordance with one embodiment of the present invention, a hair treatment composition comprises 1) a silicone grafted tapioca starch and 2) a film forming polymer. The hair treatment composition may further comprise a carrier and/or an optional ingredient. The hair treatment composition delivers in desirable root lift/volume and good hair feel combinations. Additionally, this combination minimizes visible residue that may occur with other particle types. The desirable root lift/volume, good hair feel, and low residue combinations can be demonstrated by the test method described herein. Additionally, data supporting these benefits for the composition described herein can be found in FIG. 4.

### A. SILICONE GRAFTED TAPIOCA STARCH

The silicone grafted starch is a water insoluble particle (under ambient conditions) that has been modified to improve slip or smooth feel when the starch is applied to a surface.

In one embodiment the silicone grafted starch is a silicone grafted tapioca starch that has shown the key benefits of hair feel and root lift/volume is made by Akzo Nobel. Its trade name is Dry Flo TS and its INCI name is Tapioca Starch Polymethylsilsesquioxane. It is produced by a reaction of methyl sodium siliconate (polymethylsilsesquioxane) and tapioca starch. Tapioca starch is sourced from the Cassava root by standard means know in the art. This silicone grafted tapioca starch is commercially available as CAS no. 68989-12-8. In one embodiment the tapioca starch comprises about 83% amylopectin and about 17% amylase. This is grafted to sodium methyl siliconate. The silicone grafted tapioca starch can be formed using any known means, including, but not limited to those methods described in U.S. Pat. Nos. 7,375,214, 7,799,909, 6,037,466, 2,852,404, 5,672,699, and 5,776,476.

The silicone grafted starch can be added to a hair treatment or styling composition at a level of from about 0.01% to about 5% by weight of the hair treatment composition. In one embodiment the level is from about 0.1 to about 2%.

### B. FILM FORMING MATERIAL

The silicone grafted starch can further be combined with a film forming polymer, to deliver the desired consumer benefit of lift and volume. This film forming polymer can be added to the hair treatment or styling composition at a level of from about 0.01% to about 5% by weight of the hair treatment composition. In one embodiment the level is from about 0.1% to about 2%. A film forming polymer is a large molecule typically with repeating structural units (monomers) which, when applied to a surface and cured or dried, yields a thin film on the surface. These film forming polymers may have a variety of functions within the formulation including: providing viscosity (increasing composition thickness), providing hair conditioning (smoothness, reduced friction), or by providing root lift/volume to the hair fibers. Suitable film forming polymers include, but are not limited to, Acyrlates, Acylamides, Silicones, Polyvinyl compounds, polyquaterium compounds, elastomeric materials, latexes, polyurethanes, polyethylenes, polystyrenes, nylon compound, wax based compounds as well as natural polymers such as shellac, rubber, cellulose, chitosans, polysaccarides, proteins and any combination thereof.

In another embodiment the film forming polymers include silicone compounds. Water or alcohol/water soluble compounds includes siloxane derivates with PEG or PPG or both modifications such as PEG 10 dimethicone, PEG 8 dimethicone, PEG 12 dimethicone, and any combination thereof.

In other embodiments the hair composition comprises film forming polymers which provide some degree of root lift or style. The styling film forming polymers include but are not limited to PVP's, PVP/VA, acrylates, polyurethanes, polyquaterium compounds, and polysaccharides. Tradenames for these styling polymers include but are not limited to AMAZE, Amphomer, BioStyle, Luviquat, Luviflex, Luvimer, Luviset, Ultrahold, AQ, Acudyne, Kytamer, Aqua Style, Styleze, Eco Smooth, Gantrez, Aquaflex, Advantage, Gafquat, OmniRex, and the PVP K series.

In another embodiment the hair treatment composition comprises film forming polymers which can be prepared from the free-radically polymerizable monomers vinylpyrrolidone, methacrylamide and vinylimidazole. The preparation of such a polymer is described in WO 03/092640, e.g. example Nos. 61, 62, 64 and 65, and is commercially available under the name Luviflex® Clear (BASF) (INCI name: VP/Methacrylamide/Vinyl Imidazole Copolymer). Additional suitable film forming polymers include, but are not limited to, those disclosed in US20080020004A1, published Jan. 24, 2008.

1. Silicones

In another embodiment the film forming polymers include silicone compounds. Water or alcohol/water soluble compounds includes siloxane derivates with PEG or PPG or both modifications such as PEG 10 dimethicone, PEG 8 dimethicone, PEG 12 dimethicone, and any combination thereof.

2. Viscosity Modifiers

In one embodiment, the agent according to the invention comprises at least one viscosity-modifying substance in an amount of from about 0.01 to about 20% by weight or from about 0.05 to about 10% by weight or in another embodiment from about 0.1 to about 5% by weight. In one embodiment the viscosity-modifying substance is a thickening polymer, chosen from copolymers of at least one first monomer type, which is chosen from acrylic acid and methacrylic acid, and at least one second monomer type, which is chosen from esters of acrylic acid and ethoxylated fatty alcohol; crosslinked polyacrylic acid; crosslinked copolymers of at least one first monomer type, which is chosen from acrylic acid and methacrylic acid, and at least one second monomer type, which is chosen from esters of acrylic acid with C10- to C30-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C10- to C30-alcohol and a third monomer type, chosen from C1- to C4-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C2-, C3- or C4-alkylene and styrene; polyurethanes; hydroxypropyl starch phosphate; polyacrylamide; copolymer of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate (e.g. PEG-150/stearyl alcohol/SMDI copolymer).

3. Styling Polymers

In one embodiment the film forming polymer is a hairstyling polymer selected from the group consisting of amphoteric hairstyling polymers, zwitterionic hairstyling polymers, anionic hairstyling polymers, non-ionic hairstyling polymers, cationic hairstyling polymers, and mixtures thereof.

The hairstyling formulation may comprise a cationic hairstyling polymer. Cationic hairstyling polymers suitable for the present invention may be selected from polymers with cationic or cationizable groups. Suitable cationic polymers include those with primary, secondary, tertiary or quaternary amino groups. In one embodiment the cationic charge density is from 1 to 7 meq/g. Suitable cationic polymers contain quaternary amino groups. Cationic polymers can be homo- or copolymers, where the quaternary nitrogen groups are contained either in the polymer chain or as substituents on one or more of the monomers. The ammonium group-containing monomers can be copolymerized with non-cationic monomers. Suitable cationic monomers are unsaturated compounds that can undergo radical polymerization, which bear at least one cationic group, especially ammonium-substituted vinyl monomers such as, for example, trialkylmethacryloxyalkylammonium, trialkylacryloxyalkylammonium, dialkyldiallylammonium and quaternary vinylammonium monomers with cyclic, cationic nitrogen-containing groups such as pyridinium, imidazolium or quaternary pyrrolidones, e.g. alkylvinylimidazolium, alkylvinylpyridinium, or alkylvinylpyrrolidone salts. The alkyl groups of these monomers are lower alkyl groups such, as for example, C-1 to C-7 alkyl groups, and in one embodiment are C-1 to C-3 alkyl groups.

The ammonium group-containing monomers can be copolymerized with non-cationic monomers. Suitable comonomers are, for example, acrylamide, methacrylamide, alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, for example vinyl acetate, vinyl alcohol, propylene glycol or ethylene glycol, where the alkyl groups of these monomers are C-1 to C-7 alkyl groups, and in one embodiment are C-1 to C-3 alkyl groups.

Suitable polymers with quaternary amino groups include, for example, those described in the CTFA Cosmetic Ingredient Dictionary under the designations ‘polyquaternium’ such as methylvinylimidazolium chloride/vinylpyrrolidone copolymer (polyquaternium-16) or quaternized vinylpyrrolidone/dimethylaminoethyl methacrylate copolymer (polyquaternium-11; Gafquat® 755N-PW from ISP) as well as quaternary silicone polymers or silicone oligomers such as, for example, silicone polymers with quaternary end groups (quatemium-80).

Suitable cationic polymers of synthetic origin include: poly(dimethyldiallylammonium chloride); copolymers from acrylamide and dimethyldiallylammonium chloride; quaternary ammonium polymers, formed by the reaction of diethyl sulfate with a copolymer from vinylpyrrolidone and dimethylaminoethyl methacrylate, especially vinylpyrrolidone/dimethylaminoethyl methacrylate methosulfate copolymer (e.g. Gafquat® 755 N; Gafquat® 734); quaternary ammonium polymers from methylvinylimidazolium chloride and vinylpyrrolidone (e.g. Luviquat® HM 550 from BASF; Luviquat® Hold from BASF; polyquaternium-46 [vinylcaprolactam {VCap}, vinylpyrrolidone {VP} and quaternized vinylimidazole {QVI}] from BASF; Luviquat® FC 905 from BASF [polyquaternium-16]); Luviquat Supreme® from BASF (polyquaternium-68, quaternised copolymer of vinyl pyrrolidone, methacrylamides, vinyl imidazole and quaternized vinyl imidazole); polyquaternium-35; polyquaternium-57; polymers from trimethylammonium ethyl methacrylate chloride; terpolymers from dimethyldiallylammonium chloride, sodium acrylate and acrylamide (e.g. Merquat® Plus 3300); copolymers from vinylpyrrolidone, dimethylaminopropyl methacrylamide, and methacryloylaminopropyllauryldimethylammonium chloride; terpolymers from vinylpyrrolidone, dimethylaminoethyl methacrylate, and vinylcaprolactam (e.g. Gaffix® VC 713); vinylpyrrolidone/methacrylamidopropyltrimethylammonium chloride copolymers (e.g. Gafquat® HS 100); copolymers from vinylpyrrolidone and dimethylaminoethyl methacrylate; copolymers from vinylpyrrolidone, vinylcaprolactam, and dimethylaminopropylacrylamide; poly- or oligoesters formed from at least one first type of monomer that is selected from hydroxyacids substituted with at least one quaternary ammonium group; dimethylpolysiloxane substituted with quaternary ammonium groups in the terminal positions.

Suitable cationic polymers that are derived from natural polymers include, in particular, cationic derivatives of polysaccharides, for example, cationic cellulose derivatives, starch, or guar. Cationic polysaccharides are, for example, represented by the general formula:

G-O—B—N⁺—R^{a}—R^{b}—R^{c}X⁻

G is an anhydroglucose residue, for example, starch or cellulose anhydroglucoses;

B is a divalent bonding group, for example, alkylene, oxyalkylene, polyoxyalkylene or hydroxyalkylene; R^{a}, R^{b} and R^{c} are, independently from one another, alkyl, aryl, alkylaryl, arylalkyl, alkoxyalkyl or alkoxyaryl, any of which can have up to 22 carbon atoms, wherein the total number of carbon atoms in R^{a}, R^{b} and R^{c} is in one embodiment a maximum of 20.

Cationic cellulose derivatives include those that have at least one quaternary ammonium group, e.g. a copolymer made of hydroxyethyl cellulose and diallyldimethyl ammonium chloride (polyquaternium-4), or the reaction product made of hydroxyethyl cellulose and an epoxide substituted with a trialkyl ammonium group (polyquaternium-10), wherein the alkyl groups can have 1 to 20 carbon atoms, and in one embodiment the alkyl groups are methyl groups. In one embodiment the molecular weight is between about 100,000 and about 600,000, in another embodiment the molecular weight is between about 200,000 to about 400,000. In one embodiment the nitrogen content is from about 0.5 to 4%, in another embodiment from about 1.5 to 3%. In one embodiment the cellulose derivative is polyquaternium-4, which is sold under the trade names Celquat® H100 and Celquat® L200.

Cationic latex hairstyling polymers are also suitable.

In one embodiment the cationic polymers are polyquaternium-4, polyquaternium-11, polyquaternium-16, polyquaternium-68, mixtures thereof, and mixtures of polyquaternium-68 with non-ionic polymers. In another embodiment the cationic polymers are polyquaternium-4, polyquaternium-11, polyquaternium-68, and mixtures thereof.

In an embodiment, the hairstyling formulation comprises less than about 0.1% by weight chitosan, chitosan salts and chitosan derivatives. In another embodiment, the hairstyling formulation is substantially free from chitosan, chitosan salts and chitosan derivatives.

When the aerosol hairspray product comprises from about 15% to less than about 30% VOC, suitable cationic polymers are polyquaternium-4, polyquaternium-11, polyquaternium-16, polyquaternium-68, mixtures thereof; and in one embodiment are polyquaternium-4, polyquaternium-68, and mixtures thereof.

When the aerosol hairspray product comprises from about 30% to less than about 54% VOC, suitable cationic hairstyling polymers are polyquaternium-4, polyquaternium-11, polyquaternium-68, mixtures thereof; and in one embodiment are polyquaternium-4, polyquaternium-68, and mixtures thereof.

The hairstyling formulation may comprise amphoteric or zwitterionic hairstyling polymers. Zwitterionic and amphoteric polymers may be selected from: copolymers formed from alkylacrylamide, alkylaminoalkyl methacrylate, and two or more monomers from acrylic acid and methacrylic acid as well as, if necessary, their esters, especially copolymers from octylacrylamide, acrylic acid, butylaminoethyl methacrylate, methyl methacrylate and hydroxypropyl methacrylate (octylacrylamide/acrylate/butylaminoethyl methacrylate copolymer, for example Amphomer® from Akzo Nobel); copolymers, that are formed from at least one of a first type of monomer that possesses quaternary amino groups and at least one of a second type of monomer that possesses acid groups; copolymers from fatty alcohol acrylates, alkylamine oxide methacrylate and at least one monomer selected from acrylic acid and methacrylic acid as well as, if necessary, acrylic acid esters and methacrylic acid esters, especially copolymers from lauryl acrylate, stearyl acrylate, ethylamine oxide methacrylate and at least one monomer selected from acrylic acid and methacrylic acid as well as, if necessary, their esters; copolymers from methacryloyl ethyl betaine and at least one monomer selected from methacrylic acid and methacrylic acid esters; copolymers from acrylic acid, methyl acrylate and methacrylamidopropyltrimethylammonium chloride (polyquaternium-47); copolymers from acrylamidopropyltrimethylammonium chloride and acrylates or copolymers from acrylamide, acrylamidopropyltrimethylammonium chloride, 2-amidopropylacrylamide sulfonate and dimethylaminopropylamine (polyquaternium-43); oligomers or polymers, producible from quaternary crotonoylbetaines or quaternary crotonoylbetaine esters.

Amphoteric and zwitterionic latex hairstyling polymers are also suitable.

Amphoteric polymers such as Amphomer® are present in their neutralized or partially neutralized form. Suitable neutralisers include potassium hydroxide, sodium hydroxide, triisopropanolamine (TIPA), 2-aminobutanol, 2-aminomethyl propanol (AMP), aminoethylpropandiol, dimethyl stearamine (Armeen 18 D), sodium silicate, tetrahydroxypropyl ethylenediamine (Neutrol® TE), ammonia (NH₃), triethanolamine, trimethylamine (Tris Amino Ultra), aminomethylpropandiol (AMPD), and in one embodiment are 2-aminobutanol, ammonia, and 2-aminomethyl propanol. In another embodiment the neutralizing agent for Amphomer® is 2-aminomethyl propanol.

Suitable amphoteric or zwitterionic polymers are polyquaternium-47, octylacrylamide/acrylate/butylaminoethyl methacrylate copolymers, and mixtures thereof; and in one embodiment are octylacrylamide/acrylate/butylaminoethyl methacrylate copolymers, particularly Amphomer® from Akzo Nobel.

The hairstyling formulation may comprise anionic polymers. Suitable anionic hairstyling polymers are selected from among terpolymers from acrylic acid, ethyl acrylate, and N-tert-butylacrylamide; crosslinked or uncrosslinked vinyl acetate/crotonic acid copolymers; terpolymers from tert-butylacrylate, ethyl acrylate and methacrylic acid; sodium polystyrenesulfonate; copolymers from vinyl acetate, crotonic acid and vinyl propionate; copolymers from vinyl acetate, crotonic acid and vinyl neodecanoate; aminomethylpropanol/acrylate copolymers; copolymers from vinylpyrrolidone and at least one further monomer selected from among acrylic acid, methacrylic acid, acrylic acid esters and methacrylic acid esters; copolymers from methyl vinyl ether and maleic acid monoalkyl esters; aminomethylpropanol salts of copolymers from allyl methacrylate and at least one further monomer selected from among acrylic acid, methacrylic acid, acrylic acid esters and methacrylic acid esters; crosslinked copolymers from ethyl acrylate and methacrylic acid; copolymers from vinyl acetate, mono-n-butyl maleate and isobornyl acrylate; copolymers from two or more monomers selected from among acrylic acid, methacrylic acid, acrylic acid esters and methacrylic acid esters, copolymers from octylacrylamide and at least one monomer selected from among acrylic acid, methacrylic acid, acrylic acid esters and methacrylic acid esters; polyesters from diglycol, cyclohexanedimethanol, isophthalic acid and sulfoisophthalic acid; polyurethanes; and copolymers of polyurethane and acrylates. Suitable polyester polymers include polyester-5 polymers, for example AQ® 48 Ultra Polymer, (diglycol/CHDM/isophthalates/SIP copolymer), AQ® 55 S, and AQ® 38 S, all from Eastman Chemical Company. Also suitable is a polyvinylmethacrylic acid/maleic acid copolymer (Omnirez® 2000 from ISP). Anionic latex hairstyling polymers are also suitable.

Suitable anionic polymers are selected from the group consisting of polyurethane-1 (e.g. Luviset® P.U.R. from BASF), polyurethane-14/AMP-acrylates polymer blend (e.g. DynamX® from Akzo Nobel), and mixtures thereof. In one embodiment the anionic polymer is polyurethane-1.

When the aerosol hairspray product comprises from about 41% to about 54% VOC, suitable anionic polymers are selected from the group consisting of polyurethane-14/AMP-acrylates polymer blend (e.g. DynamX® from Akzo Nobel), polyester-5 polymers, (e.g. AQ® 48 Ultra Polymer, AQ® 55 S, AQ® 38 S, all from Eastman Chemical Company), and mixtures thereof.

In one embodiment the anionic polymers are present in their neutralized or partially neutralized form. Suitable neutralisers include potassium hydroxide, sodium hydroxide, triisopropanolamine (TIPA), 2-aminobutanol, 2-aminomethyl propanol (AMP), aminoethylpropandiol, dimethyl stearamine (Armeen 18 D), sodium silicate, tetrahydroxypropyl ethylenediamine (Neutrol® TE), ammonia (NH₃), triethanolamine, trimethylamine (Tris Amino Ultra), aminomethylpropandiol (AMPD), and in one embodiment 2-aminobutanol, ammonia, and 2-aminomethyl propanol. In another embodiment the neutralizing agent is 2-aminomethyl propanol.

The hairstyling formulation may comprise a non-ionic hairstyling polymer. Suitable non-ionic polymers include, but are not limited to, homo- or copolymers that are formed from at least one of the following monomers: vinylpyrrolidone, vinylcaprolactam, vinyl esters such as, for example, vinyl acetate, vinyl alcohol, acrylamide, methacrylamide, alkyl- and dialkylacrylamide, alkyl- and dialkylmethacrylamide, alkyl acrylate, alkyl methacrylate, propylene glycol or ethylene glycol, where the alkyl groups in these monomers are C-1 to C-7 alkyl groups, and C-1 to C-3 alkyl groups. Suitable homopolymers are, for example, those of vinylcaprolactam, vinylpyrrolidone or N-vinylformamide. Further suitable non-ionic hairstyling polymers include copolymers from vinylpyrrolidone and vinyl acetate, terpolymers from vinylpyrrolidone, vinyl acetate and vinyl propionate, polyacrylamides; polyvinyl alcohols as well as polyethylene glycol/polypropylene glycol copolymers. Also suitable are polyvinylpyrrolidone/dimethylaminopropylaminoacrylates copolymer (Aquaflex® SF 40 from ISP); isobutylene ethylmaleinimide/hydroxy ethylmaleinimide copolymer (Aquaflex® FX 64 from ISP); vinylcaprolactam/polyvinylpyrrolidone/dimethylaminoethyl methacrylate copolymer (Advantage® from ISP). Non-ionic latex hairstyling polymers are also suitable.

Suitable non-ionic polymers include polyvinylpyrrolidone (PVP); polyvinylpyrrolidone/vinylacetate (PVP/VA) copolymers; polyvinylpyrrolidone, polyvinylcaprolactam, vinylpyrrolidone/vinyl acetate copolymers; copolymers of vinylpyrrolidone, methacrylamide and vinylimidazole; polyvinyl alcohol, isobutylene/ethylmaleimide/hydroxyethylmaleimide copolymers; and copolymers from vinylpyrrolidone, vinyl acetate and vinyl propionate. In one embodiment the non-ionic polymers include polyvinylpyrrolidone (K90, 85, 80, 60, 30), polyvinylpyrrolidone/vinyl acetate copolymers (PVP/VA 64), terpolymers of vinylpyrrolidone, methacrylamide and vinylimidazole (e.g. Luviset® Clear from BASF), and mixtures thereof. In another embodiment the non-ionic polymers include PVP K 60, 30, and PVP/VA 37/64. In yet another embodiment the non-ionic polymers include PVP K60 and PVP/VA 37/64.

When the aerosol hairspray product comprises from about 40% to about 54% VOC, suitable non-ionic polymers are: polyvinylpyrrolidone polymers, and in one embodiment vinylpyrrolidone/vinyl acetate copolymers (PVP/VA), and in another embodiment PVP/VA 37.

The hairstyling formulation may comprise latex hairstyling polymers. As used herein, a “latex hairstyling polymer solution” is a droplet of latex hairstyling polymer in water. The advantage of latex polymer solutions is that they have a similar viscosity to water, even when the composition comprises up to 50% by weight latex polymer. Water thin polymer solutions can be sprayed easily without clogging the valve-insert combination and still provide good hold.

Latex hairstyling polymers are selected from the group consisting of anionic latex hairstyling polymers, amphoteric latex hairstyling polymers, non-ionic latex hairstyling polymers, cationic latex hairstyling polymers, and mixtures thereof.

Suitable anionic latex polymers include urethane-based polymers, for example polyurethane-34 (Baycusan® from Bayer). Polyurethane-34 is described in EP2105127A1. In an embodiment of the present invention, the hairstyling polymer is the latex hairstyling polymer polyurethane-34. Suitable amphoteric latex hairstyling polymers include latex resins based on styrene/butylacrylate or methylmethacrylate/butylacrylate latex mixed with Amphomer® and AMP (EP0688557B 1). Suitable non-ionic latex hairstyling polymers include styrene-butadiene polymers, acrylic, vinyl acetate polymers, and mixtures thereof, with non-ionic ethylene oxide/propylene oxide block copolymer surfactants (U.S. Pat. No. 5,525,657). Suitable cationic latex hairstyling polymers include cationic graft-modified rubber latex polymers with a cationic and/or non-ionic surfactant (U.S. Pat. No. 6,512,034).

The hairstyling formulation may comprise blends of hairstyling polymers. When there is a blend of a cationic hairstyling polymer combined with an anionic hairstyling polymer then the cationic hairstyling polymer in one embodiment is than about 0.2%, in another embodiment less than about 0.15%, and in another embodiment less than about 0.1%, by weight of the hairstyling formulation and propellant. Suitable polymer combinations include the following: cationic cellulose derivatives from hydroxyethyl cellulose and diallyldimethylammonium chloride in conjunction with vinylpyrrolidone/vinyl acetate copolymers; chitosan in conjunction with polyvinylpyrrolidone; quaternary ammonium polymers from methylvinylimidazolium chloride and vinylpyrrolidone in conjunction with vinylpyrrolidone/vinyl acetate copolymers and/or polyvinylpyrrolidone; and octylacrylamide/acrylates/butylaminoethyl methacrylate copolymer (Amphomer®) with polyvinylpyrrolidone (PVP).

In one embodiment the polymer combinations are: vinylpyrrolidone/vinyl acetate copolymers (PVP/VA), and polyvinylpyrrolidone (PVP); and octylacrylamide/acrylates/butylaminoethyl methacrylate copolymer (Amphomer®) with polyvinylpyrrolidone (PVP).

### C. THICKENER

In one embodiment the film forming polymers include cosmetic ingredient thickeners to increase the substantivity of the composition, such that it does not drip undesirably onto other areas of the body, clothing or home furnishings. Any suitable thickener can be used, for example, a cellulose based thickener such as hydroxypropylmethylcelluloses. In another embodiment, suitable thickeners such as the carbopol and carbopol derivatives options from Lubrizol, Ultrez 10, Ultrez 20, and Ultrez 21 as well as the acrylamide options from Seppic like the Sepigel line are included in the composition. Additional suitable examples of thickening polymers are Xanthan gum or Xanthan gum derivatives like Dehydroxanthan gum (Amaze XT from Akzo Nobel).

### D. CARRIER

The cosmetic, hair-setting or hair care polymers usually used for these purposes exhibit good setting and/or care properties in aqueous, alcoholic or aqueous-alcoholic media. In a particular embodiment, the composition comprises from about 10-90% alcohol, alternatively from about 15-75% alcohol, or alternatively from about 25-50% alcohol. Any suitable alcohol, such as ethanol, can be used. However, in another embodiment the composition is free of alcohol, and the carrier is water. In this embodiment the composition is from about 10% to about 99.5% water, in another embodiment from about 50% to about 99% water, and from yet another embodiment from about 75% to about 99% water.

However, all requirements such as, for example, feel, shine, combability, durability of setting etc. may not be satisfied. WO 03/092640 discloses water-soluble copolymers with (meth)acrylamide units and their use in hair-treatment agents. SÖFW-Journal, 12-2003, page 65-72 describes hairstyling compositions which comprise a vinylpyrrolidone/methacrylamide/vinylimidazole copolymer.

### E. OPTIONAL INGREDIENTS

The hair treatment and/or styling compositions of the present invention can also additionally comprise any suitable optional ingredients as desired. Such optional ingredients should be physically and chemically compatible with the components of the composition, and should not otherwise unduly impair composition stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

The compositions of the present invention can include a xanthine compound. As used herein, “xanthine compound” means one or more xanthines, derivatives thereof, and mixtures thereof. Xanthine Compounds that can be useful herein include, but are not limited to, caffeine, xanthine, 1-methyl xanthine, theophylline, theobromine, derivatives thereof, and mixtures thereof. In one embodiment, the composition comprises from about 0.1% to about 10% of a xanthine compound, in another embodiment from about 0.5% to about 5% of a xanthine compound, and in yet another embodiment from about 1% to about 2% of a xanthine compound.

The compositions of the present invention can include a vitamin B3 compound. Vitamin B3 compounds are particularly useful for regulating skin conditions, as described in U.S. Pat. No. 5,939,082. In some embodiments, the composition comprises from about 0.1% to about 25% of a vitamin B3 compound, in another embodiment from about 0.5% to about 15% of a vitamin B3 compound, and in yet another embodiment from about 3.5% to about 7.5% of a vitamin B3 compound. As used herein, “vitamin B3 compound” means a one or more compounds having the formula:

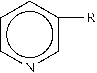

wherein R is —CONH₂ (i.e., niacinamide), —COOH (i.e., nicotinic acid) or —CH2OH (i.e., nicotinyl alcohol); derivatives thereof; mixtures thereof; and salts of any of the foregoing.

Exemplary derivatives of the foregoing vitamin B3 compounds include nicotinic acid esters, including non-vasodilating esters of nicotinic acid (e.g, tocopherol nicotinate, myristyl nicotinate), nicotinyl amino acids, nicotinyl alcohol esters of carboxylic acids, nicotinic acid N-oxide and niacinamide N-oxide.

Suitable esters of nicotinic acid include nicotinic acid esters of C₁-C₂₂, in one embodiment C₁-C₁₆, and in another embodiment C₁-C₆ alcohols. The alcohols are suitably straight-chain or branched chain, cyclic or acyclic, saturated or unsaturated (including aromatic), and substituted or unsubstituted. In one embodiment the esters are non-vasodilating. As used herein, “non-vasodilating” means that the ester does not commonly yield a visible flushing response after application to the skin in the subject compositions (the majority of the general population would not experience a visible flushing response, although such compounds may cause vasodilation not visible to the naked eye, i.e., the ester is non-rubifacient). Non-vasodilating esters of nicotinic acid include tocopherol nicotinate and inositol hexanicotinate; tocopherol nicotinate can be used.

Other derivatives of the vitamin B₃ compound are derivatives of niacinamide resulting from substitution of one or more of the amide group hydrogens. Nonlimiting examples of derivatives of niacinamide useful herein include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., C1-C18). Specific examples of such derivatives include nicotinuric acid (C8H8N2O3) and nicotinyl hydroxamic acid (C6H6N2O2), which have the following chemical structures:

### Nicotinuric Acid:

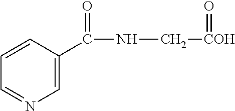

### Nicotinyl Hydroxamic Acid:

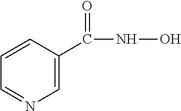

Exemplary nicotinyl alcohol esters include nicotinyl alcohol esters of the carboxylic acids salicylic acid, acetic acid, glycolic acid, palmitic acid and the like. Other non-limiting examples of vitamin B3 compounds useful herein are 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, and niaprazine.

Examples of the above vitamin B3 compounds are well known in the art and are commercially available from a number of sources, e.g., the Sigma Chemical Company (St. Louis, Mo.); ICN Biomedicals, Inc. (Irvin, Calif.) and Aldrich Chemical Company (Milwaukee, Wis.).

One or more vitamin B3 compounds may be used herein. In one embodiment the vitamin B3 compounds are niacinamide and/or tocopherol nicotinate.

In embodiment when used, salts, derivatives, and salt derivatives of niacinamide include those having substantially the same efficacy as niacinamide.

The compositions of the present invention may comprise a panthenol compound. As used herein, the term “panthenol compound” is broad enough to include panthenol, one or more pantothenic acid derivatives, and mixtures thereof. Panthenol and its derivatives can include D-panthenol ([R]-2,4-dihydroxy-N-[3-hydroxypropyl)]-3,3-dimethylbutamide), DL-panthenol, pantothenic acids and their salts, the calcium salt, panthenyl triacetate, royal jelly, panthetine, pantotheine, panthenyl ethyl ether, pangamic acid, pantoyl lactose, Vitamin B complex, or mixtures thereof. Additional optional ingredients include those described in US Publication 2008/0059313A1 published on Mar. 6, 2008.

Additional optional ingredients include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, (e.g. hydroquinone, kojic acid, ascorbic acid, magnesiuim ascorbyl phosphate, ascorbyl glucoside, pyridoxine), skin-conditioning agents (e.g. humectants and occlusive agents), skin treating agents (e.g. vitamin D compounds, mono-,di-, and tri-terpenoids, beta-ionol, cedrol), thickeners, hair conditioning agents, and surfactants.

In one embodiment, the composition can comprise at least one nitrone derivative. Nitrones are capable of irreversibly capturing electrons and/or free radicals, thereby reducing the relative amount of oxidative potential in a microenvironment. These can include α-phenyl butyl nitrone (PBN), PBN doxylcyclohexane radicals, 5,5-dimethylpyrroline N-oxide (DMPO), α-(4-pyridyl 1-oxide)-N-tert-butylnitrone (POBN), 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, α-(4-pyridyl-1-oxide)-N-t-butylnitrone, 3,3,5,5-tetramethylpyrroline N-oxide, and 2,4,6-tri-t-butylnitrosobenzene, or spin-trapping derivatives thereof, and mixtures thereof. In a particular embodiment, the spin trap is PBN.

The compositions of the present invention may also contain an anti-dandruff agent. Suitable, non-limiting examples of anti-dandruff particulates include: pyridinethione salts, azoles, selenium sulfide, particulate sulfur, and mixtures thereof. Such anti-dandruff particulate should be physically and chemically compatible with the essential components of the composition, and should not otherwise unduly impair product stability, aesthetics or performance.

Pyridinethione anti-dandruff particulates, especially 1-hydroxy-2-pyridinethione salts, are one embodiment of a particulate anti-dandruff agents for use in compositions of the present invention. The concentration of pyridinethione anti-dandruff particulate typically ranges from about 0.1% to about 4%, by weight of the composition. In an embodiment, the concentration of pyridinethione anti-dandruff particulate ranges from about 0.1% to about 3%, and in a further embodiment, ranges from about 0.3% to about 2%. In an embodiment of the present invention, pyridinethione salts include those formed from heavy metals such as zinc, tin, cadmium, magnesium, aluminum and zirconium. In an embodiment of the present invention, a pyridinethione salts formed from a heavy metal zinc, and in a further embodiment, the zinc salt of 1-hydroxy-2-pyridinethione (known as “zinc pyridinethione” or “ZPT”), and yet a further embodiment of 1-hydroxy-2-pyridinethione salts in platelet particle form, wherein the particles have an average size of up to about 20μ. In an embodiment of the present invention, the particles have an average size up to about 5μ, and in a further embodiment up to about 2.5μ. Salts formed from other cations, such as sodium, may also be suitable. Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. It is contemplated that when ZPT is used as the anti-dandruff particulate in the compositions herein, that the growth or re-growth of hair may be stimulated or regulated, or both, or that hair loss may be reduced or inhibited, or that hair may appear thicker or fuller.

In addition to the anti-dandruff active selected from polyvalent metal salts of pyrithione, the present invention may further comprise one or more anti-fungal or anti-microbial actives in addition to the metal pyrithione salt actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. In an embodiment of the present invention, anti-microbials include itraconazole, ketoconazole, selenium sulphide and coal tar.

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01% to about 5%. In an embodiment of the present invention, the azole anti-microbial active is included in an amount from about 0.1% to about 3%, and in a further embodiment, from about 0.3% to about 2%, by weight of the composition. In an embodiment of the present invention, the azole anti-microbial is ketoconazole.

Selenium sulfide is a particulate anti-dandruff agent suitable for use in the anti-microbial compositions of the present invention, effective concentrations of which range from about 0.1% to about 4%, by weight of the composition, and in an embodiment of the present invention, from about 0.3% to about 2.5%, and in a further embodiment from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), and in an embodiment of the present invention, less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

Sulfur may also be used as a particulate anti-microbial/anti-dandruff agent in the anti-microbial compositions of the present invention. Effective concentrations of the particulate sulfur are typically from about 1% to about 4%, by weight of the composition, and in an embodiment of the present invention from about 2% to about 4%.

The present invention may further comprise one or more keratolytic agents such as Salicylic Acid.

Additional anti-microbial actives of the present invention may include extracts of melaleuca (tea tree) and charcoal. The present invention may also comprise combinations of anti-microbial actives. Such combinations may include octopirox and zinc pyrithione combinations, pine tar and sulfur combinations, salicylic acid and zinc pyrithione combinations, octopirox and climbasole combinations, and salicylic acid and octopirox combinations, zinc pyrithione and climbasole and mixtures thereof. These actives, when used herein, are used at levels of from about 1% to about 4%, and in an embodiment of the present invention, from about 2% to about 4%.

### F. COMPOSITION FORM

In one embodiment, the agent according to the invention is in the form of an O/W emulsion, a W/O emulsion or a microemulsion.

In one embodiment, the agent according to the invention is in the form of a spray composition, either in combination with a mechanical pump spray device or in combination with at least one propellant chosen from propane, butane, dimethyl ether and fluorinated hydrocarbons. An aerosol spray can additionally comprise about 15 to about 85% by weight, particularly about 25 to about 75% by weight, of a propellant and is bottled in a pressurized container. Suitable propellants are, for example, lower alkanes, such as, for example, n-butane, isobutane and propane, or mixtures thereof, and dimethyl ether or fluorinated hydrocarbons, such as F 152a (1,1-difluoroethane) or F 134 (tetrafluoroethane), and also propellants which are in gaseous form at the pressures under consideration, such as, for example, N2, N2O and CO2, and mixtures of the abovementioned propellants.

A nonaerosol hairspray is sprayed with the help of a suitable mechanically operated spraying device. Mechanical spraying devices are understood as meaning those devices which permit the spraying of a composition without use of a propellant. A suitable mechanical spray device used may, for example, be a spray pump or an elastic container provided with a spray valve in which the cosmetic agent according to the invention is bottled under pressure, where the elastic container expands and from which the agent is continuously dispensed as a result of the elastic container contracting upon opening the spray valve.

In one embodiment, the agent according to the invention is in the form of a foamable composition (mousse) in combination with a devices for foaming, comprises at least one customary foam-imparting substance known for this purpose, e.g. at least one foam-forming surfactant or at least one foam-forming polymer. Devices for foaming are understood as meaning those devices which enable the foaming of a liquid with or without use of a propellant. A suitable mechanical foaming device which can be used is, for example, a standard commercial pump foamer or an aerosol foaming head. The composition is either in combination with a mechanical pump foaming device (pump foam) or in combination with at least one propellant (aerosol foam) in an amount of from about 1 to about 20% by weight, in another embodiment from about 2 to about 10% by weight. Propellants are chosen, for example, from propane, butane, dimethyl ether and fluorinated hydrocarbons. The agent is foamed directly prior to application and incorporated into the hair as foam and can then be rinsed out or left in the hair without rinsing.

The foamable compositions comprise, as active ingredients or additives, in one embodiment, polymers which are chosen from chitosan, chitosan salts, chitosan derivatives, cationic cellulose compounds, copolymers of vinylpyrrolidone, vinylcaprolactam and a basic acrylamide monomer or mixtures of these polymers. Suitable chitosan salts, chitosan derivatives, cationic cellulose derivatives are, for example, those mentioned above. Suitable cationic cellulose compounds include, but are not limited to, copolymers of hydroxyethylcellulose and diallyldimethylammonium chloride (polyquaternium-4) and reaction products of hydroxyethylcellulose and epoxides substituted by a trialkylammonium group (Polyquaternium-10). Suitable chitosan salts include, but are not limited to, the salts with formic acid, lactic acid and pyrrolidonecarboxylic acid. Suitable copolymers of vinylpyrrolidone, vinylcaprolactam and a basic acrylamide monomer are those in which the acrylamide monomer is dimethylaminopropylacrylamide. One embodiment of foamable compositions comprises copolymers of hydroxyethylcellulose and diallyldimethylammonium chloride (polyquaternium-4) and copolymers of vinylpyrrolidone, vinylcaprolactam and dimethylaminopropylacrylamide, and foamable compositions which comprise copolymers of hydroxyethylcellulose and diallyldimethylammonium chloride (polyquaternium-4) and copolymers of vinylpyrrolidone, vinylcaprolactam and dimethylaminopropylacrylamide, and at least one chitosan salt.

In one embodiment, the agent according to the invention is in the form of a hair wax, i.e. it has a wax-like consistency and comprises at least one of the above-mentioned waxes in an amount of from about 0.5 to about 30% by weight, and optionally further water-insoluble substances. In one embodiment the wax-like consistency has a needle penetration number (unit of measurement 0.1 mm, test weight 100 g, test time 5 s, test temperature 25° C.; according to DIN 51 579) of greater than or equal to about 10, and in another embodiment greater than or equal to about 20 and that the solidification point of the composition is greater than or equal to about 30° C. and less than or equal to about 70° C., and in one embodiment in the range from about 40 to about 55° C. Suitable waxes and water-insoluble substances are, in particular, emulsifiers with an HLB value below 7, silicone oils, silicone waxes, waxes (e.g. wax alcohols, wax acids, wax esters, and in particular natural waxes such as beeswax, carnauba wax, etc.), fatty alcohols, fatty acids, fatty acid esters or hydrophilic waxes, such as, for example, high molecular weight polyethylene glycols with a molecular weight of from about 800 to about 20 000, and in one embodiment from about 2000 to about 10 000 g/mol.

If the hair-treatment agent according to the invention is in the form of a hair lotion, then it is in the form of an essentially nonviscous or low-viscosity, flowable solution, dispersion or emulsion with a content of at least about 10% by weight, in another embodiment from about 20 to about 95% by weight, of a cosmetically compatible alcohol. Alcohols which can be used are, in particular, the lower alcohols having 1 to 4 carbon atoms customarily used for cosmetic purposes, e.g. ethanol and isopropanol.

If the hair-treatment agent according to the invention is in the form of a hair cream, then it can be in the form of an emulsion and comprises either additionally viscosity-imparting ingredients in an amount of from about 0.1 to about 10% by weight, or the required viscosity and creamy consistency is built up in a customary manner through micelle formation with the help of suitable emulsifiers, fatty acids, fatty alcohols, waxes etc.

The examples below serve to illustrate the subject matter of the invention in more detail. Unless stated otherwise, the polymer contents given in each case refer to the solids content.

### G. TEST METHODS

1. Root Lift Test

Acquire hair switches with the following characteristics:
a) 4″ total length, crimped and taped at end. Crimp/tape extends 1″ from end of switch.
b) Flat crimp approximately ⅜″ by 3/16″.
c) Brown Caucasian virgin hair.
d) 3″ of hair (uncrimped product application area) weighs ˜1.44 g.
   Switch Treatment: In 73F/45RH CT room:
a) Soak switch in purified and distilled water.
b) Remove switch from water, squeegee excess water from switch using fingers.
c) Pat switch with paper towel to remove excess surface water.
d) Apply 0.4 ml of test product (0.2 ml per side) at top of switch (just below crimp) and work product through switch with gloved fingers.
e) Bundle switch by pulling down switch to create a round switch profile.
f) Hang switch on cart with crimped end held in clamp and uncrimped end directly below clamp.
g) Leave cart in 73F/45RH CT room until ready to run test.

### Switch Testing:

a) Allow treated switches to dry in 73F/45RH CT room at least overnight.
b) Remove switch from clamp, using coarse side of standard barber's comb, comb through switch three times pulling from top (crimped) of switch through bottom (un-crimped) of switch. If higher hold product's are being evaluated, additional combing will better differentiate results. Record combing protocol on results if it deviates from control (3× coarse) protocol.
c) Place combed switch in clamp, clamped end at top, on Switch cart. Bottom of crimp/tape should be aligned with bottom of clamp. Orient switch such that if switch has natural curvature along length, the concave side of switch is on the left side as you face the switches. Un-crimped end of switch is at the bottom of the switch.
d) Remove cart from 73F/45RH CT room and place in ambient conditions.
e) Invert switches by rotating clamp approximately 180 degrees. Crimped end of switch is now at the bottom of the switch. Adjust the angle of the clamp such that the top left edge of the un-crimped end is just to the right of the left edge of the crimped (clamped) end. This is done to insure that all of the switches fall to the right as you face the cart.
f) Place cart (containing switches) into 80F/80RH CT Room, record time.
g) At 30+/−5 minutes, return to room with camera (Coolpix P500 or similar). Using 20″ stick as guide, position front camera lens ˜20″ from bar holding clamp, with camera directly in front of clamp. Take picture (auto setting, default lens length). Repeat this procedure for all switches.
h) Repeat step #7 at 60, 90 and 120 minutes.

### Switch Cart:

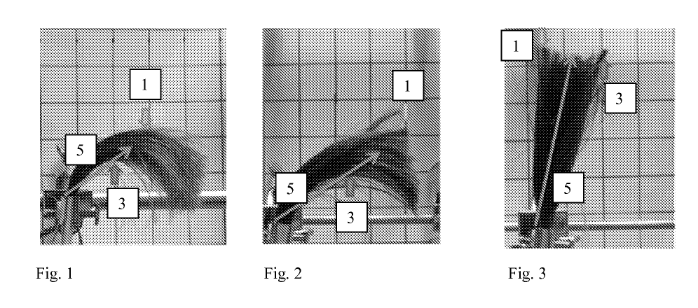

The switch cart has 5 rows holding 4 clamps per row (20 total). The clamps are designed to spin freely until clamped so hair can be hung straight down and rotated to straight up. Behind the bar rows is a 1″ by 1″ grid covering the area behind all the samples (and clamps).
The height metrics are generated by:
a) The 1″ grid covering the area behind all the samples superimposes the switch onto an XY plot. This allows various points on the switch to be assigned an XY value, estimating to nearest ½″.
b) As the switches are placed to fall to the right, the left side of the switch is always the maximum height. The maximum height of the left side of the switch (designated MAXY @ time) is measured from the top left side of the switch clamp to the maximum height of the left side of the switch on the Y axis, estimating to nearest ½″. As actual switch length varies slightly, the maximum allowable length is 4.5″ (even if it estimates to 5″).
c) The other height metric, designated “ARC” is shown in FIGS. 1-3. The high point 1 defined as maximum “Y” on the left side of the switch is shown in FIGS. 1-3. The high point 3 is defined as maximum “Y” on the right side of the switch is shown in FIGS. 1-3. The Arc length 5 is the midpoint of measured points, and is shown in FIGS. 1-3.
d) The ARC metric is the length of the line in the illustration above. As can be seen in the illustration, the switch to the left has curled over and generates the shortest arc metric (line). The middle switch has only a slight increase in the maximum switch height over the switch to left but generates an arc metric almost twice as long. In this way, we capture the increase in lift demonstrated as less switch curl is observed.

### Additional Information:

a) The switches are hung on the cart in a random order, not by the evaluator, the results are blind until the test is completed.
b) At least two switches are treated with the same product. Results are averaged to produce composite scores.
c) Higher numbers for both MAXY and ARC indicate better root lift.

2. Residue and Qualitative Feel Score
a) After completion of the height metric images taken at the two hour mark in the 80F/80RH CT Room, the switches are inverted so that they hang straight down with clamped end at top of switch. The cart is removed from the 80F/80RH CT Room into ambient conditions and allowed to equilibrate for at least 30 minutes prior to evaluating feel.
b) Prior to evaluating feel, each switch is examined for “residue”. Residue is defined as visible material remaining on the hair switch after bulk of product has dried (generally white in appearance), The switches are scored for residue as 0=no visible residue, 1=low amount, 2=medium amount, 3=high amount. The results for each like treated switch is averaged to create a composite residue score.
c) Test subjects are asked to evaluate each switch in the test for “Smooth Feel” and “Cross Fiber Friction”. The test directions are:

Directions:
1) Evaluate all switches for Smooth Feel: pull fingers down switch from near top to just past middle by squeezing the switch between the thumb and forefinger with low to moderate constant pressure, do not evaluate end of switch. (Damage causes poor feel on all switches at tip.) Excellent would represent very easy pull down with high hair alignment. A poor score will feel rough, or poorly aligned and tangled hair. 5 pt SCALE (both metrics): 1=Excellent, 2=Very Good, 3=Good, 4=Fair, 5=Poor
2) Record the results for each switch. It is ok to go back and forth or feel all first to get a sense of excellent, good and poor.
3) After evaluating and recording the smooth feel score, evaluate for Cross Fiber Friction “CFF” by rubbing across hair in the middle of the switch. Record results using the same 5 pt scale as the smooth feel. Squeeze switch at midpoint between thumb and forefinger with low to moderate pressure. With low to moderate constant pressure. Roll switch back and for the between fingers.
4) For Cross Fiber Friction, a score of Excellent represents hair that slides easily past each other as single fibers. Clumps or hairs not wanting to slide over each other easily will score higher numbers.
5) Between 6 and 12 test subjects generate the feel scores per test. The average Smooth Feel and CFF scores are generated per switch along with a composite score representing the average of the Smooth Feel and CFF average scores. These scores are then averaged across all switches with same treatment.

### H. EXAMPLES

| | All ingredients in % as added | | | | | |
|---|---|---|---|---|---|---|
| | Leave on Treatment Composition Examples | | | | | |
| | | | Leave on | | | |
| | Leave on | Leave on | Treatment | Spray | Styling | Styling |
| Ingredient | Treatment 1 | Treatment 2 | Conditioner | on Gel | Gel | Mousse |
| Water | QS | QS | QS | QS | QS | QS |
| SD Alcohol 40 ¹ | 50.0000 | 50.0000 | | | | |
| Polyacrylamide & C13-14 | | | 1.0000 | | | |
| Isoparaffin & Laureth-7 ² | | | | | | |
| Acrylates/C10-30 Alkyl | 0.3500 | | | | | |
| Acrylate Crosspolymer ³ | | | | | | |
| Dehydroxanthan Gum ⁴ | | 0.7500 | | | | |
| PVP K30 ⁵ | | | | 2.00 | | |
| Chitosan ⁶ | | | | 0.700 | | |
| Carbomer K ⁷ | | | | | 1.500 | |
| VA/Croto nates Copolymer ⁸ | | | | | 1.500 | |
| Polyquaternium 4 ⁹ | | | | | | 1.8302 |
| Tapioca Starch | 0.2500 | 0.2500 | 0.2500 | 0.7500 | 1.0000 | 0.5000 |
| Polymethylsilsesquioxane ¹⁰ | | | | | | |
| Bis-PEG/PPG-20/20 Dimethicone ¹¹ | | 0.3000 | | | | |
| PEG-20/PPG-23 Dimethicone ¹² | 1.0000 | | | | | |
| 10,000 cSt Dimethicone 13 | | | 3.5000 | | | |
| SORBITOL ¹⁴ | | | | | 6.000 | |
| PROPYLENE GLYCOL ¹⁵ | | | | | | 0.4580 |
| Caffeine ¹⁶ | 0.7500 | 0.9375 | | | | |
| Niacinamide ¹⁷ | 2.5000 | 3.1250 | | | | |
| D-Panthenol ¹⁸ | 0.1500 | 0.1875 | | | | |
| PEG-40 Hydrogenated Castor Oil ¹⁹ | | | | 0.400 | 0.200 | |
| Polysorbate 20 ²⁰ | | | | 0.400 | | |
| PEG-60 Almond Glycerides ²¹ | | | | 0.200 | | |
| Polysorbate 80 ²² | | | | 0.200 | | |
| C9-11 Pareth 8 ²³ | | | | | | 0.2290 |
| Propane & Iso-Butane ²⁴ | | | | | | 6.0000 |
| Fragrance | 0.7000 | 0.3000 | 1.0000 | 0.7000 | 0.5000 | 0.7000 |
| Preservatives, pH adjusters | Up to 2% | Up to 2% | Up to 2% | Up to 2% | Up to 2% | Up to 2% |

¹ SD Alcohol 40B (200 Proof) supplier Equistar Chemicals
² Sepigel 305, 45% active, Supplier Seppic Inc.
³ Ultrez 21, 100% active, Supplier Lubrizol Inc.
⁴ Amaze XT, 100% active, Supplier Akzo Nobel
⁵ Luviskol K30, 100% active, Supplied by BASF Corp.
⁶ Poly +, 100% active, Supplied by Primex EHF
⁷ Carbomer 980 Polymer, 100% active, Supplied by Lubrizol Advanced Materials Inc.
⁸ Luviset CA 66, 100% active, Supplier BASF SE
⁹ Celquat LV, 100% active, Supplier Akzo Nobel
¹⁰ Dry Flo TS, 100% Active, Supplier Akzo Nobel
¹¹ Abil B 8832, 100% Active, Supplier Evonik Industries AG
¹² Silsoft 430, 100% Active, Supplier Momentive Inc.
13 TSF451 1MA, 100% Active, Supplier Dow Corning Toray Co Ltd
¹⁴ Karion F, 70% active, Supplier Merck KGAA
¹⁵ Propylene Glycol USP, 100% active, Supplier Dow Chemical Co.
¹⁶ Caffeine USP, 100% active, Supplier BASF Pharmachemikalien Gmbh Kg
¹⁷ Niacinamide USP FCC, 100% active, Supplier DSM Nutritional Products Inc
¹⁸ D-Panthenol, 100% active, Supplier DSM Nutritional Products Inc
¹⁹ Emulsionante ELH 49, 100% active, Supplier Erca SPA
²⁰ Tween 20, 100% active, Supplier Croda Inc.
²¹ Crovol A-70, 100% active, Supplier Croda Inc.
²² Tween 80, 100% active, Supplier Croda Inc.
²³ Neodol 91-8,100% active, Supplier Shell Chemical Co.
²⁴ Aeron A-70, 100% active, Supplier Diversified CPC International

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair treatment composition comprising:
(a) from about 0.01% to about 5% by weight of the composition of a silicone grafted tapioca starch;
(b) from about 0.01 to about %5 by weight of the composition of a film forming polymer;
(c) a carrier.

2. The hair treatment composition of claim 1, wherein the level of silicone grafted tapioca starch is from about 0.1 to about 2% by weight of the composition.

3. The hair treatment composition of claim 1, wherein the level of film forming polymer is from about 0.1 to about 2% by weight of the composition.

4. The hair treatment composition of claim 1, wherein the film forming polymer is selected from the group consisting of acrylates, acylamides, silicones, polyvinyl compounds, polyquaterium compounds, elastomeric materials, latexes, polyurethanes, polyethylenes, polystyrenes, nylon compound, and wax based compounds.

5. The hair treatment composition of claim 1, wherein the film forming polymer is a naturally derived film forming polymer selected from the group consisting of shellac, rubber, cellulose, chitosans, polysaccarides, and proteins.

6. The hair treatment composition of claim 1, wherein the composition further comprises xanthan gum.
